(19) United States
(12) Patent Application Publication
(10) Pub. No.: US 2005/0049796 A1
(43) Pub. Date: Mar. 3, 2005

(54) Methods for encoding non-biological information on microarrays

(76) Inventors: Peter G. Webb, Menlo Park, CA (US); Joseph P. Fredrick, Palo Alto, CA (US); Jacqueline M. Tso, Los Gatos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/817,115

(22) Filed: Apr. 2, 2004

Related U.S. Application Data
(63) Continuation-in-part of application No. 10/655,477, filed on Sep. 3, 2003.

(51) Int. Cl. C12Q 1/68; G06F 19/00; G01N 33/48; G01N 33/50

(52) U.S. Cl. 702/20; 435/6

(57) ABSTRACT

Methods and compositions for encoding and decoding array information on an array are provided. The methods involve contacting an array containing one or more array information features with a sample containing target that binds to at least one of the one or more array information features to produce at least one signal that provides information about the array.

### BACKGROUND OF THE INVENTION

In nucleic acid sequencing, mutation detection, proteomics, and gene expression analysis, there is a growing emphasis on the use of high density arrays of immobilized nucleic acid or polypeptide probes. Such arrays can be prepared by a variety of approaches, e.g., by depositing biopolymers, for example, cDNAs, oligonucleotides or polypeptides on a suitable surface, or by using photolithographic techniques to synthesize biopolymers directly on a suitable surface. Arrays constructed in this manner are typically formed in a planar area of between about 4-100 mm², and can have densities of up to several thousand or more distinct array members per cm².

In use, an array surface is contacted with a sample containing labeled target analytes (usually nucleic acids or proteins) under conditions that promote specific, high-affinity binding of the analytes in the sample to one or more of the probes present on the array. The goal of this procedure is to quantify the level of binding of one or more probes of the array to labeled analytes in the sample. Typically, the analytes in the sample are labeled with a detectable label such as a fluorescent tag, and quantification of the level of fluorescence associated with a bound probe represents a direct measurement of the level of binding. In turn, this measurement of binding represents an estimate of the abundance of a particular analyte in the sample. A variety of biological and/or chemical compounds may be used as detectable labels in the above-described arrays (See, e.g., Wetmur, J. Crit Rev Biochem and Mol Bio 26:227, 1991; Mansfield et al., Mol Cell Probes. 9:145-56, 1995; Kricka, Ann Clin Biochem. 39:114-29, 2002).

Such arrays are commonly used to perform nucleic acid hybridization assays. Generally, in such a hybridization assay, labeled single-stranded analyte nucleic acid (e.g., polynucleotide target) is hybridized to an immobilized complementary single-stranded nucleic acid probe. Complementary nucleic acid probe binds the labeled target polynucleotide, and the presence of the labeled target polynucleotide of interest is detected and quantified.

Arrays may be physically labeled (e.g., with a barcode) to provide a means by which information about an array can be obtained. In most cases, the array label provides a unique key that allows a user to look up information regarding the array in a database. In performing an array assay, a labeled array is incubated with a sample under specific binding conditions, and data, corresponding to the binding pattern of targets in the sample to the probes on the array, is obtained. The data obtained from an array assay is usually matched with information about an array using the label that is physically attached to the array, and the data is analyzed. While this system is commonly in use today, it has drawbacks because there are limitations in the current methods for labeling arrays.

For example, many arrays are physically labeled with a barcode which is not human readable. In the absence of the barcode, a barcode reader, or a database of array information with a key corresponding to the barcode, the array information corresponding to the array may not be identifiable. Also, once an array has been scanned, the array, including the label that is physically attached to the array, is usually discarded. As such, if the array label is incorrect, or if the array label is not read or read incorrectly, it may be impossible, after the time at which an error was made, to correctly associate array information with any data for the array. Furthermore, since the array label is usually affixed to only one position on a substrate that often contains multiple arrays, the label may provide information about each array on the substrate.

As such, improved methods of providing information about arrays are needed. This invention meets this, and other, needs.

### SUMMARY OF THE INVENTION

Methods and compositions for encoding and decoding array information on an array are provided. The methods involve contacting an array containing one or more array information features with a sample containing target that binds to at least one of the one or more array information features to produce at least one signal that provides information about the array. In many embodiments the signal is a symbol or a code, such as binary-code or non-binary-code, that provides the information about the array. In certain embodiments, the array information is typically decoded using a file containing decoding information. Kits and systems are provided for performing the invention. The methods can be used in a variety of applications, for example gene expression analysis, DNA sequencing, mutation detection and other genomics, as well as other proteomics applications.

### BRIEF DESCRIPTION OF THE DRAWINGS

FIGS. 1 is a composite figure showing six schematic representations of exemplary embodiments of the invention, A-F.

FIG. 2 is an image of a microarray showing exemplary results of the invention.

FIG. 3. schematically illustrates an embodiment of the invention

FIG. 4. schematically illustrates an embodiment of the invention

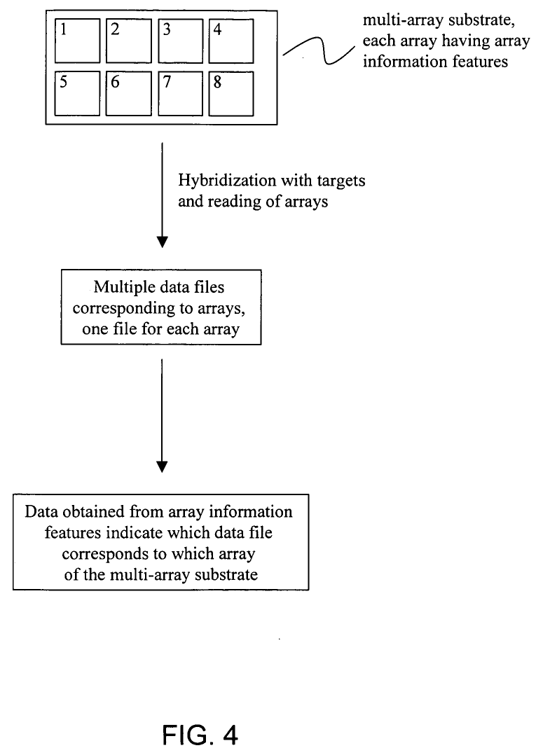

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles.

In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

A “biopolymer” is a polymeric biomolecule of one or more types of repeating units.

Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups, one or both of which may have removable protecting groups).

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimics of such biopolymeric binding agents, etc. In many embodiments of interest, the arrays are arrays of nucleic acids, including oligonucleotides, polynucleotides, cDNAs, mRNAs, synthetic mimics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm2 or even less than 10 cm2. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Arrays on the surface of a multi-array substrate are usually independently contactable with sample. In other words, in the absence of any cross-contamination, the arrays may each be separately incubated with sample under conditions suitable for specific binding of targets in the sample with the probes on the arrays. The arrays on the surface of a multi-array substrate are independently contactable with sample because they are spatially distinct, i.e., are physically separated by a distance or structure, that allows different samples to be independently applied to each array of the substrate and then incubated.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which pre-made probes are immobilized on a substrate surface, immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026. The surface of a substrate may be treated with an organosilane coupling agent to functionalize the surface. One exemplary organosilane coupling agent is represented by the formula RₙSiY₍₄₋ₙ₎ wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group that can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

Also, instead of drop deposition methods, light directed fabrication methods may be used, as are known in the art. Inter-feature areas need not be present particularly when the arrays are made by light directed synthesis protocols.

Where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other). Target nucleic acids are found in a sample. The identity of the target nucleotide sequence generally is known to an extent sufficient to allow preparation of various probe sequences hybridizable with the target nucleotide sequence. The term “target sequence” refers to a sequence with which a probe will form a stable hybrid under desired conditions. The target sequence generally contains from about 30 to 5,000 or more nucleotides, preferably about 50 to 1,000 nucleotides. The target nucleotide sequence is generally a fraction of a larger molecule or it may be substantially the entire molecule such as a polynucleotide as described above. The minimum number of nucleotides in the target nucleotide sequence is selected to assure that the presence of a target polynucleotide in a sample is a specific indicator of the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g. transcription of a DNA template into RNA) and the efficiency of detection and/or amplification of the target nucleotide sequence, where appropriate.

A “probe” is a chemical moiety, e.g., a biopolymer that is usually immobilized on a substrate, and forms a feature, or element, on an array. Probes, like targets, may be nucleic acids, antibodies, polypeptides, and the like. Nucleic acid probes are hybridizable in that they have a nucleotide sequence that can hybridize to a target nucleic acid, if present, under suitable hybridization conditions. In most embodiments, a probe is a single stranded nucleic acid of at least about 15 bp, at least about 20 bp, at least about 30 bp, at least about 50 bp, at least about 100 bp, at least about 200 bp, at least about 500 bp, at least about 800 bp, at least about 1 kb, at least about 1.6 kb, at least about 2kb, at least about 3kb or at least about 5 kb or more in length.

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1 % SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50.° C. or about 55° C. to about 60° C.; or, a salt concentration of about 0.15 M NaCl at 72° C. for about 15 minutes; or, a salt concentration of about 0.2×SSC at a temperature of at least about 50° C. or about 55.° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1 % SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37.° C. (for 14-base oligos), 48.° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned. with a T(U), A, C, and G, respectively, of the other sequence. RNA sequences can also include complementary G=U or U=G base pairs. Thus, two sequences need not have perfect homology to be “complementary” under the invention, and in most situations two sequences are sufficiently complementary when at least about 85% (preferably at least about 90%, and most preferably at least about 95%) of the nucleotides share base pair organization over a defined length of the molecule.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

The term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Information about an array” or “array information” as will be described in greater detail below, refers to information that is particular to an array, such as, e.g., an unique identifier for an array or for a batch of arrays with which further information about an array may be obtained using a database, the identifier that makes each array of a multi-array substrate unique (e.g., arrays on a multi-array substrate may be labeled 1-8, for example), information about the structure of an array, such as the comers of an array, the orientation of an array, or elements of interest on an array (which may be provided by means of a “pointer” encoded on the array), or information about the probes in an array, such as the species from which the probes are derived, or whether the probes are oligonucleotide probes or cDNA probes. In particular embodiments, “array information” conveys information to data analysis software regarding how data obtained from an array may be analyzed. Once array information is obtained, data analysis software, in view of the information, may analyze data obtained from an array in a particular way. For example, array information may indicate which diseases or conditions an array may be used to investigate or diagnose. That information may be used by data analysis software to analyze data obtained from that array to obtain information about any or all of those diseases.

Array information is distinct from sample or target information because array information yields no relevant information about a sample or targets, except for targets that bind to the array information features, present in a sample. Mere binding of a target to a feature on an array provides no information about the array unless the feature is part of set of one or more features for providing information about the array.

An “one or more array information features” of an array, as will be discussed in greater detail below, represents one or more features, which, when present in an array, provides information about the array, usually when at least one of the array information features is bound by a labeled target. Array information features are usually present in a set of “one or more” array information features that contains at least one, or possibly more than one, array information features.

An array information feature usually contains an “array information probe”. A plurality of array information features may contain only one array information probe if the array information features all contain the same probe. As such, a single array information probe may be present in a plurality of features.

Information about an array may be “encoded” in data obtained from an array, if that data is obtained from one or more array information features contained in that array. Information may be encoded using any suitable encoding system, e.g., any alphabet, including the English and Braille alphabets, or binary or non-binary coding systems, for example.

Encoded information may be “decoded”, i.e., translated from one form of code to another, by any suitable decoding system. Typically, encoded information is decoded to provide a human or computer readable version of the information. For example, a binary code (e.g., a binary coded decimal) may be decoded to provide an Arabic number or the like.

The term “using” is used herein as it is conventionally used, and, as such, means employing, e.g. putting into service, a method or composition to attain an end. For example, if a program is used to create a file, a program is executed to make a file, the file usually being the output of the program. In another example, if a file is used, it is usually accessed, read, and the information stored in the file employed to attain an end. Similarly if a unique identifier, e.g. a barcode is used, the unique identifier is usually read to identify, for example, an object or file associated with the unique identifier.

A unique identifier is a unique code (e.g. a number) that is “associated” with an object or file. If a unique identifier is associated with an object, the object is usually labeled with the unique identifier. For example, the unique identifier may be written on an object, or the unique identifier may be contained on a the surface of a label (e.g., a paper or plastic label) which is adhered to the object. In certain embodiments, the unique identifier is a barcode, and the barcode, as is known in the art, is usually present on the surface of a label that is adhered to the object. As is known in the art, there are several ways of associating a file with a unique identifier. For example, the file may be named with the unique identifier, the file may contain the unique identifier embedded in the file, e.g., as a file header, or the file may have a file path that is unique to the file, and the file path uniquely indicates the file.

Binding of a probe to a target may be “evaluated”. “Evaluated”, in this context, means that the presence, absence or level of binding of the probe to the target is determined or assessed. Binding of a probe to a target may be evaluated absolutely, e.g., in the absence of binding data for a target to another probe, or relatively, e.g. relative to binding of the probe or another probe to another target. As such, no numerical figure need be associated with the binding of a target to a probe in order for the binding to be evaluated. Accordingly, evaluation may be qualitative, quantitative or semi-quantitative.

### DETAILED DESCRIPTION OF THE INVENTION

Methods and compositions for encoding and decoding array information on an array are provided. The methods involve contacting an array containing one or more array information features with a sample containing target that binds to at least one of the one or more array information features to. produce at least one signal that provides information about the array. In many embodiments the signal is a symbol or a code, such as binary-code or non-binary-code, that provides the information about the array. The array information is typically decoded using a file containing decoding information. Kits and systems are provided for performing the invention. The methods can be used in a variety of applications, for example gene expression analysis, DNA sequencing, mutation detection and other genomics, as well as other proteomics applications.

Before embodiments of the present invention are described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items. are provided solely for their disclosure prior to the filing date of the present application. Nothing herein is to be construed as an admission that the present invention is not entitled to antedate such material by virtue of prior invention. Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, compositions for use in methods of providing information about an array are described first, followed by a description of the subject methods. Applications in which the subject methods find use are then described, followed by a description and of kits for use in practicing the subject methods.

Compositions

The invention provides a system for providing information about an array. The system, in general, involves an array containing one or more array information features, and a target that specifically binds to at least one of the one or more array information features to provide information about the array. These components of this system will be described separately and in greater detail below.

Array Information Features

Array information features are regions of an array that contain array information probes. In general, array information features are usually present as one or more array information features in an array. In most embodiments, array information features make up less than about 5% (e.g., less about 0.5%, less than about 1%, less than about 3%), usually no more than up to about 10% of the total number of elements or features in a single array. In a single array, therefore, there may be 1, 2, about 4 or more, about 8 or more, about 12 or more, about 16 or more, about 48 or more, about 96 or more, about 192 or more, including up to 384 or more, array information features. Each of these features may contain a single array information probe, two or more array information probes (e.g., two, three or four array information probes), or in some embodiments, no probe. As such, an individual array information feature, e.g., one spot on an array, may contain 0, 1, or a mixture of 2, 3, or 4 or more probes. In exemplary embodiments where a single array information probe is used, a subset of the array information features usually contains the probe, whereas the remainder of the features usually do not contain the array information probe. In these embodiments, it is the presence or absence of a probe in particular array identification elements that provides information about an array. In other exemplary embodiments where two array identification probes are used, each of the array information features usually contains one or both of the probes. In these embodiments, if the array information features each contain a single probe, it is the presence or absence of the probes in particular array identification elements that provides information about an array. Similarly, in embodiments where two probes are present in a single array information feature, it is usually the relative abundance of the probes that provides information about an array.

Typically, an array information probe, if present in an array information feature, will not detectably hybridize under stringent conditions to targets other than complementary array information targets in a sample. Suitable array information probes may be selected, for example, by generating test array information probes and testing them in silica, e.g., by using BLAST or any other sequence comparison program to determine if the test array information probe is likely to bind to a test array information target, or, for example, by generating test array information probes and testing them experimentally, e.g., by performing binding assays (for example, hybridization assays) to determine if the array information probe binds to a chosen target. Suitable array information probes may also be selected if a suitable array information target has already been identified: a suitable array information probe will normally have a sequence that is complementary to the sequence of a suitable target.

As such, a suitable array information probe may have a known or unknown sequence, or a specific or random sequence, depending on how the array information probe is selected. In some embodiments, particularly those in which information is provided using a two array information probes, the array information probes usually have a sequence that is not present in the genome of an organism represented by the non-array-information probes on an array. In other words, in some embodiments, if an array contains probes for genes and gene products of a specific species, e.g., humans, the array information probes on the array will have a sequence that is not represented in the genome of that species or its gene products. For example, in embodiments where the sample contains targets derived from a human, an array information probe may be from yeast, bacteria or any other organism, or may have any other sequence, such that it will not specifically bind to targets in a sample from humans.

In other embodiments, particularly embodiments in which information is provided using a single array information probe, the array information probe may have a sequence that is designed or selected to bind to a targets in a sample from a particular species. In embodiments that use samples derived from humans, a suitable array information probe may be a probe for a constitutively expressed gene product, such as a products of a glyceraldehydes-3-phosphate dehydrogenase, a mitochondrial ATPase, ubiquitin, or actin gene, that is constitutively expressed in humans.

Array information features may be positioned in an array at any suitable location. In certain embodiments, array information features may be positioned so that they form a defined pattern, such as a recognizable symbol, e.g., a letter of the alphabet, a number, a letter of a non-English alphabet, a pictogram, a picture, an icon or a word, and, as such, they are usually positioned proximal to each other in the array. Such symbols or words are usually written using a “dot matrix”, which is a well known system for writing symbols using a series of dots. Recognizable symbols may also be represented by any suitable system, including the Braille alphabet, in which each unit of the Braille alphabet is represented by six dots in a 2 by 3 dot matrix.

In certain embodiments, array information features are positioned at the corners or sides of an array. For example, array information features indicating the corners of an array are usually placed at the four corners of an array. In certain other embodiments, particularly embodiments in which the array information features provided encoded information, the array information features may be positioned at any pre-determined positions on an array. For example, the array information features that are part of a set of eight array information features may each be situated at a different position on the array. In certain embodiments, however, array information elements that provide encoded information are usually situated adjacent to one other, usually in a horizontal or vertical line.

In certain embodiments, particularly those embodiments in which array information features provide a non-binary code, an individual array information feature may contain a mixture of two or more probes at pre-determined relative concentrations. Depending on the methods used, probes may be mixed together in multiples of any suitable ratio (e.g., 1/4, 1/8, 1/10, 1/12, 1/16, 1/26, and the like). For example, if methods involving decimal code (in which all numbers may be represented by only ten numerals) are used, individual array features may contain two probes at ratios of 1:10, 2:5, 3:10, 2:5, 1/2, 6/10, 7/10, 4/5, 9/10 or 1:1, or, alternatively, at ratios of 1:1, 2:1, 3:1, 4:1, 5:1, 6:1, 7:1, 8:1, 9:1 or 10:1.

Array Information Targets

Array information targets usually specifically bind to a single corresponding (i.e., complementary) array information probe. In many embodiments, an array information target does not detectably bind to other targets in the sample in which it is present or to probes other than a corresponding array information probe. Typically, array information targets do not detectably hybridize to probes other than array information probes, and are distinguishable from analyte targets, for which estimates of their abundance in the sample are desirable.

As with the array information probes, suitable array information targets may be selected based on their complementarity to a suitable probe, or by any other means such as the in silica or experimental methods described above for selecting a suitable array information target. Also like array information probes, array information probes may have a known or unknown sequence, or a specific or random sequence, depending on how the array information target is selected.

In general, an array information target has a sequence that is complementary to array information a probe, and, as such, will bind to the probes under specific binding conditions.

As discussed above, in most embodiments, one or two or more probes (e.g., 2, 3, 4, 5 or 6 or more probes that are present singly or mixed) are used to make one or more one array information features on an array. In general, the number of array information targets used in the subject methods corresponds to the number of different array information probes. In other words, if the methods involve one array information probe, and that array information probe is present in, for example, eight elements, the methods will generally use one array information target since one array information target is sufficient to detect the array information probe in all eight elements. Similarly, if there are two array information probes used in the subject methods, the methods will use two array information targets that correspond to those probes.

In most embodiments, array information targets are labeled independently of the rest of the targets of a sample, and are spiked (i.e., added or mixed) into the sample prior to use. One or two labeled array information targets are usually spiked into a sample prior to contacting of the sample with an array.

For example, array information targets may be labeled using a T7 RNA amplification labeling procedure and stored, each labeled array information target in a separate tube. As needed, desired volume (usually about 1-5 μl) of a labeled array information targets is usually aliquoted the storage tube into a sample tube and mixed with the analyte sample, prior to application of the sample onto an array. Array information targets may be added to a tube prior to, at the same time as, or after the addition of an analyte sample to a tube.

Array information targets may be labeled using any known labeling methods. Methods for labeling proteins and nucleic acids are generally well known in the art (e.g. Brumbaugh et al Proc Natl Acad Sci USA 85, 5610-4, 1988; Hughes et al. Nat Biotechnol 19, 342-7, 2001, Eberwine et al Biotechniques. 20:584-91, 1996, Ausubel, et al, Short Protocols in Molecular Biology, 3rd ed., Wiley & Sons, 1995 Sambrook, et al, Molecular Cloning: A Laboratory Manual, Third Edition, 2001 Cold Spring Harbor, N.Y. and DeRisi et al. Science 278:680-686, 1997; Patton W F. Electrophoresis. 2000 21:1123-44; MacBeath G. Nat Genet. 2002 32 Suppl:526-32; and Biotechnol Prog. 1997 13:649-58). These means usually involve either direct chemical modification of the analyte, or a labeled nucleotide that is incorporated into a nucleic acid by nucleic acid replication, e.g., using a polymerase.

Chemical modification methods for labeling a nucleic acid sample usually include incorporation of a reactive nucleotide into a nucleic acid, e.g., an amine-allyl nucleotide derivative such as 5-(3-aminoallyl)-2′-deoxyuridine 5′-triphosphate, using an RNA-dependent or DNA-dependent DNA or RNA polymerase, e.g., reverse transcriptase or T7 RNA polymerase, followed by chemical conjugation of the reactive nucleotide to a label, e.g. a N-hydroxysuccinimdyl of a label such as Cy-3 or Cy5 to make a labeled nucleic acids. Such chemical conjugation methods may be combined with RNA amplification methods, to produce labeled DNA or RNA.

Suitable labels may also be incorporated into a sample by means of nucleic acid replication, where modified nucleotides such as modified deoxynucleotides, ribonucleotides, dideoxynucleotides, etc., or closely related analogues thereof, e.g. a deaza analogue thereof, in which a moiety of the nucleotide, typically the base, has been modified to be bonded to the label. Modified nucleotides are incorporated into a nucleic acid by the actions of a nucleic acid-dependent DNA or RNA polymerases, and a copy of the nucleic acid in the sample is produced that contains the label. Methods of labeling nucleic acids with radioactive or non-radioactive tags by a variety of methods, e.g., random priming, nick translation, RNA polymerase transcription, etc., are generally well known in the art (e.g., Ausubel, et al, Short Protocols in Molecular Biology, 3rd ed., Wiley & Sons, 1995 and Sambrook, et al, Molecular Cloning: A Laboratory Manual, Third Edition, 2001 Cold Spring Harbor, N.Y.).

Labels of interest include directly detectable and indirectly detectable radioactive and non-radioactive labels such as fluorescent dyes. Directly detectable labels are those labels that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels.

Fluorescent labels that find use in the subject invention include a fluorophore moiety. Specific fluorescent dyes of interest include: xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G ⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc.

In certain embodiments, the labels used in the subject methods are distinguishable, meaning that the labels can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, OR), BODIPY V-1002 and BODIPY V1005 (Molecular Probes, Eugene, Oreg.), POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002).

As discussed above, in making a labeled array information target, it is generally desirable to label the target in a single reaction tube, and then add a portion of the labeled array information target to a sample prior to its incubation with an array.

Methods

Also provided are methods for obtaining information about an array. In general, the methods involve contacting an array containing one or more array information features with a sample that contains a target that binds to at least one of the one or more array information features to provide at least one signal, i.e., a signal from a radioactive or non-radioactive label, that provides information about the array. Array information is then provided by assessing or evaluating binding of a target to the one or more array information features, either qualitatively or quantitatively, including semi-quantitatively. In most embodiments, the presence, absence or level of probe in each array information feature, as detected by a labeled target for the probe, is assessed or evaluated, e.g., determined, and an array information target/feature binding pattern is produced. It is the pattern of binding of an array information target to the one or more array information probes that provides the array information. In certain embodiments, the information is encrypted information, e.g., information that is ciphered or changed in order to conceal its meaning. In these embodiments, encrypted information may be obtained by the subject methods, and then decrypted such that the information may be understood by a user.

Binding of an array information target to the one or more array information probes provides array information by producing a pattern of binding. As discussed briefly above, the pattern of binding may provide a defined pattern, such as a letter, word or number, or string of the same, written using any suitable such as a dot matrix or Braille system. For example, a binding pattern showing a numeral may indicate the array number of an array on a multi-array substrate, a binding pattern showing a string of letters (e.g., Hs or Sc, etc.) may indicate the species represented on the array (e.g., Homo sapiens or Saccharomyces cerevisiae), a binding pattern showing the word “control” may indicate that the array is a control array, and a binding pattern showing a string of numbers and/or letters may provide a unique identifier for the array, or a unique identifier for a batch of arrays, with which a user may use as a key to access further information about the array (e.g., the identity and position of the set of probes that are on the array).

In other embodiments, the binding pattern of an array information target to the one or more array information features provides a binary or non-binary code. For binary codes, as is well known, information is provided by a string of“0”s and “1”s in a particular order. Any number, letter or string of the same can be represented by a binary code. For example, the number 10222343, which could represent an eight digit identifier for an array, may be represented by the standard binary code number “100110111111101100000111”. In another example of a binary code, as is known in the art, decimal numbers may be represented using a binary coded decimal (BCD) system. In BCD, a string of four binary digits (0 or 1) represents each decimal number (0-9) using the standard binary code. Each digit of a decimal number can therefore be represented by a group of four binary numbers. For example, the number 10222343 could be represented by the BCD number “00010000000100010001001101000011”, where the left-most four digits represents “1”, the second four digits represents “0”, the third four digits represents “2”, and so on. In another example of a well known binary code, any string of numbers or letters may be represented by binary ASCII code. In this example, the string “Homo sapiens 10222343”, which could represent the species represented on an array and a identifier for the array, is represented by the ASCII code: “010010000110111101101101011011110010000001110011011000010111000001101001 0110010101101110011100110010000000110001001100000011001000110010001100100 01100110011010000110011”.

As discussed above, a binary code may be represented on an array by one or more array information features in which an individual feature either contains, or does not contain an array information probe. In certain embodiments, therefore, one digit of the binary code (e.g., “0”) may be indicated by the presence of an array information probe, whereas the other digit of the binary code (e.g., “1”) may be indicated by the presence of a different array information probe. For example, if two different distinguishably labeled array information targets are used, the presence of one target (as determined by the signal from its label) can represent the “0” condition and the presence of the other target (as determined by the signal from its label) can represent the “1” condition. In other words, each specific target sequence may be distinguishably labeled and specific to a complementary probe sequence on the array.

In certain other embodiments, one digit of the binary code is indicated by the absence of an array information probe and the other digit of the binary code is indicated by the presence of an array information probe. As mentioned above, the presence of these probes in an array information feature is detected using one or more array information targets.

In certain embodiments, the binding pattern of an array information target to one or more array information probes may provide a non-binary code, which, as is known in the art, is a code that has a base of any number greater than 2. Exemplary non-binary codes include octal (base 8), hexadecimal (base 16) or decimal (base 10) codes, and, in some embodiments, a base 26 code. The digits of these codes are usually represented by mixing two array information probes together in a ratio that corresponds to the desired digit. For example, the decimal code number “10222343” is represented by eight elements, each containing a probe that is present at a certain amount in relation to a control probe. In this embodiment, the number 10222343 may be represented by elements with the following probe compositions: 0A:1B (the ratio is 0),1A:1B (the ratio is 1), 2A:1B (the ratio is 2), 3A:1B (the ratio is 3) and 4A:1B (the ratio is 4), up to 9A:1B (the ratio is 9) where the ratio reflects the amount of probe A, as compared to the amount of probe B, where the amount of probe B stays at a constant level. Octal and hexadecimal codes may also be represented using a similar system, where the base number determines the number of increments for each ratio. For example, using an octal code in the above example, probe A would vary with respect to probe B in eight increments (e.g., 1:1, 2:1, etc., up to 8:1) and using a hexadecimal code in the above example, probe A would very with respect to probe B in sixteen increments (e.g., 1:1, 2:1, etc., up to 16:1).

Other non-binary or binary codes may be produced by a set of array information features when they are detected by 3 or more (e.g., 4, 5, 6, 7, 8 or more, 12 or more, usually up to about 16 or 20) distinguishably labeled array information targets. In these embodiments, the features, when bound to target, may produce a series of signals corresponding to the different labels of the probes to provide the information. For example, four array information features may be detected with four different distinguishably labeled probes to produce a series of signals of different wavelengths to provide the code. In other words, a code could be provided by a series of signals of different wavelengths, e.g., wavelengths corresponding to the wavelengths of fluorescent dyes used to label an information target. Conceptually, the code could be in the form of a series of colors, e.g., red-green-blue-yellow, where each color corresponds to a signal of a particular wavelength.

As long as the code being used is known and a user can determine the presence or relative abundance of a probe in an array information element, a digit in a binary or non-binary code can be provided. In some embodiments, a code may provide information by itself (e.g., by providing name or number that is meaningful without reference to any other information source), or may be a key, e.g., a unique identifier for an array or batch of arrays, that can be utilized to look-up information about an array in separate information source, e.g., a database.

In particular embodiments, the code being used is an error correcting code that allows for an error in at least one bit (e.g., one digit) of the code. Such error correcting codes are well known in the art and are described in the following books: Theory of Information Encoding by Robert McEliece (Cambridge University Press; 2nd edition, May 2002), The Art of Error Correcting Coding by Robert H. Morelos-Zaragoza (John Wiley & Sons; April 2002) and Error Control Coding: From Theory to Practice by Peter Sweeney (John Wiley & Sons; (May 13, 2002). In particular embodiments, the code used is a Hamming or Reed-Solomon coded.

In practicing the subject methods of this embodiment, the first step is typically to contact a sample, which in many embodiments is at least suspected to have (if not known to include) an analyte of interest, with an array of binding agents that includes a binding agent (ligand) specific for the analyte of interest under conditions sufficient for the analyte to bind to its respective binding pair member that is present on the array. Thus, if the analyte of interest is present in the sample, it binds to the array at the site of its complementary binding member and a complex is formed on the array surface. Depending on the nature of the analyte(s), the array may vary greatly, where representative arrays are reviewed in the Definitions section, above. Of particular interest are nucleic acid arrays, where in situ prepared nucleic acid arrays are employed in many embodiments of the subject invention.

To contact the sample with the array, the array and sample are brought together in a manner sufficient so that the sample contacts the surface immobilized ligands of the array. As such, the array may be placed on top of the sample, the sample may be placed, e.g., deposited on the array surface, the array may be immersed in the sample, etc.

Following contact of the array and the sample, the resultant sample contacted or exposed array is then maintained under conditions sufficient and for a sufficient period of time for any binding complexes between members of specific binding pairs to occur. In many embodiments, the duration of this step is at least about 10 min long, often at least about 20 min long, and may be as long as 30 min or longer, but often does not exceed about 72 hours. The sample/array structure is typically maintained at a temperature ranging from about 40 to about 80, such as from about 40 to 70° C. Where desired, the sample may be agitated to ensure contact of the sample with the array.

In the case of hybridization assays, the substrate supported sample is contacted with the array under stringent hybridization conditions, whereby complexes are formed between target nucleic acids that are complementary to probe sequences attached to the array surface, i.e., duplex nucleic acids are formed on the surface of the substrate by the interaction of the probe nucleic acid and its complement target nucleic acid present in the sample. An example of stringent hybridization conditions is hybridization at 50° C. or higher and 0.1 ×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, followed by washing the filters in 0.1×SSC at about 65° C. Hybridization involving nucleic acids generally takes from about 30 minutes to about 24 hours, but may vary as required. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Once the incubation step is complete, the array is typically washed at least one time to remove any unbound and non-specifically bound sample from the substrate, generally at least two wash cycles are used. Washing agents used in array assays are known in the art and, of course, may vary depending on the particular binding pair used in the particular. assay. For example, in those embodiments employing nucleic acid hybridization, washing agents of interest include, but are not limited to, salt solutions such as sodium, sodium phosphate and sodium, sodium chloride and the like as is known in the art, at different concentrations and may include some surfactant as well.

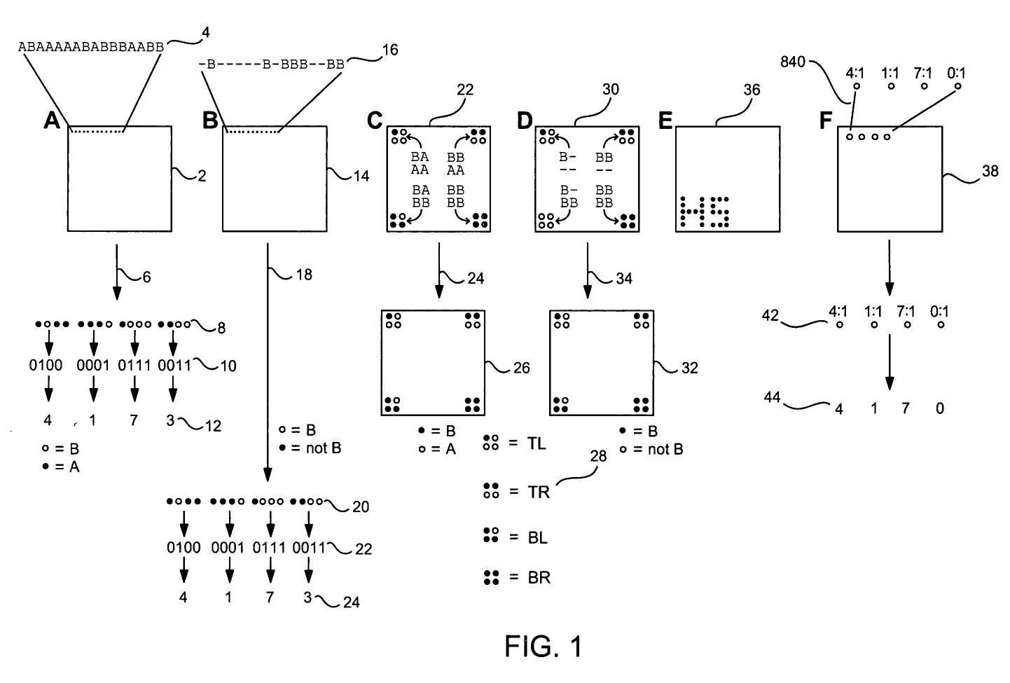

FIGS. 1A-1F shows six exemplary embodiments of the invention, A-F. In each of the embodiments shown in these figures, an array is provided that contains a set of array information features. The positioning of the array information features, the type of code or symbols used to convey information, the content of the array information elements and the content of the information to be conveyed is usually pre-determined prior to making the array. In some embodiments, the information for an array may be present in a database. In these embodiments, a unique identifier for that information may be used as the information to be conveyed by the subject methods. In order to provide a set of array information features, information (e.g., corresponding to a unique key in a database) may be first encoded into binary or non-binary codes prior to placing the one or more array information features corresponding to those codes on an array.

The following description references the exemplary embodiments illustrated in FIGS. 1A-1F. It is not intended that the invention should be limited to the embodiments showing in this figure. Upon description of the embodiments illustrated in FIGS. 1A-1F, other embodiments that are not specifically described in the figures will become apparent to one of skill in the art.

In a first embodiment shown in FIG. 1A, an array 2 containing a set of array information features 4 of probe compositions A or B is hybridized 6 with array information targets complementary to probes A and B. After hybridization of the array information targets to array information features, the binding of the array information targets to the array information features is assessed to provide a binding pattern 8, in which a filled circle represents binding of probe A and an open circle represents binding of probe B. Conversion of this binding pattern to a binary code, where binding of A represents “0” and binding of B represents “1”, provides a binary code 10, which, when converted into decimal code is the number “4173” 12, which represents information about the array.

In a second embodiment shown in FIG. 1B, an array 14 containing a set of array information features 16 of probe compositions “B” and “−”, i.e. a probe that is not B, is hybridized 18 with an array information target complementary to probe B. After hybridization of the array information target to array information features, the binding of the array information target to array information features is assessed to provide a binding pattern 20, in which a filled circle represents no binding, and an open circle represents binding of probe B. Conversion of this binding pattern to a binary code, where no significant probe binding is “0” and binding of B represents “1”, provides a binary code 22, which, when converted into decimal code is the number “4173” 24, which represents information about the array.

In a third embodiment shown in FIG. 1C, an array 22 containing a set of array information features containing probes A or B at each corner of the array is hybridized 24 with array information targets complementary to probes A and B. After hybridization of the array information targets to the array information features, the binding of the array information targets to the array information features is assessed to provide a binding pattern 26, where binding of A is represented by an open circle and binding of B is represented by a filled circle. The pattern may be interpreted using a key 28, where certain binding patterns are associated with the top right (TR), top left (TL), bottom left (BL) and bottom right (BR) comers of the array.

In a fourth embodiment shown in FIG. 1D, an array 30 containing a set of array information features containing probe B or not containing B, i.e., “−”, at each corner is hybridized 34 with an array information target complementary to probe B. After hybridization of the array information target to the sets of array information features, the binding of the array information target to the array information features is assessed to provide a binding pattern 32, where no binding is represented by an open circle and binding of B is represented by a filled circle. Again, the pattern may be interpreted using a key 28 where certain binding patterns are associated with the top right (TR), top left (TL), bottom left (BL) and bottom right (BR) corners of the array.

In a fifth embodiment shown in FIG. 1E, an array 36 containing a set of array information features that are situated on the array such that they form the letters “H” and “S” is hybridized with an array information that binds to those elements. After hybridization of the array information target to the sets of array information features, the binding of the array information target to array information features is assessed to provide a binding pattern, shown in array 36, in which the letters “H” and “S” are shown. The letters provide information about the array.

In a sixth embodiment shown in FIG. 1F, an array 40 containing a set of array information features, each containing a mixture of probes A and B at predetermined concentrations 40 in which probe A is present at a varying concentration compared to a constant amount of probe B. After hybridization of array information targets complementary to probes A and B to the array, the binding of probes A and B is assessed to provide a series of ratios 42 that correspond to the relative concentrations of the individual array information probes in an array information feature. Converted into decimal code, those ratios represent the number 4173, which provide information about the array.

In most embodiments, the presence of any binding complexes on the array surface is detected, e.g., through use of a signal production system, e.g., an isotopic or fluorescent label present on the analyte, etc. In other words, the resultant array is interrogated or read to detect the presence of any binding complexes on the surface thereof, e.g., the label is detected using colorimetric, fluorimetric, chemiluminescent or bioluminescent means. The presence of the analyte in the sample is then deduced or determined from the detection of binding complexes on the substrate surface.

Utility

The present invention finds use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. In these assays, an array containing one or more array information features is usually hybridized under specific binding conditions with a sample containing a labeled target nucleic acid that binds at least one of the one or more array information features, and at least one complex between the target nucleic acids and the probes contained in the features is formed. The presence of hybridized complexes is then detected, and, in many embodiments, information about the array is obtained by analyzing these hybridization complexes. Specific hybridization assays of interest which may be practiced using the arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Specific hybridization assays of interest which may be practiced using the subject arrays include: genomic hybridization, gene discovery assays, differential gene expression analysis assays; nucleic acid sequencing assays, mutation detection, and the like. The subject compositions and methods find particular use in assays that involve multi-array substrates and in assays for which information about an array is desirable. The subject methods allows a user to obtain information about an array independently from the information provided by a barcode or other label physically associated with an array. Upon obtaining information about an array, a user may, for example, cross-compare the obtained information to the label information in order to verify the identity of the array, assign any data obtained from the array to a particular array, or view any data obtained from the array without looking up information using the label physically associated with the array.

Where the arrays are arrays of polypeptide binding agents, e.g., protein arrays, specific applications of interest include analyte detection/proteomics applications, including those described in: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the methods include a step of transmitting information, e.g., data or an array information decoding system, from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical, light, or any other signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read, following a wash. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

The subject methods may be incorporated into any current array assay by using set of one or more array information features and targets for those features to provide information about an array.

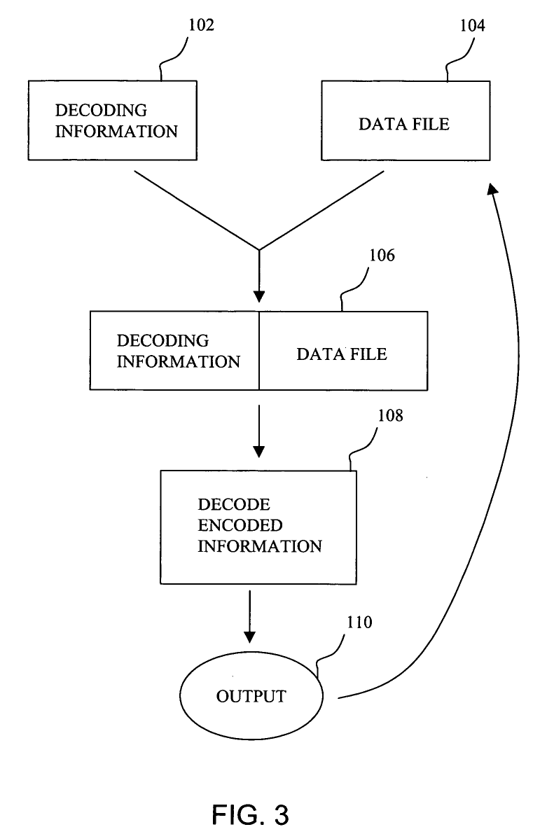

In particular embodiments, the invention finds use in indicating an identifier of an array of a multi-array substrate. As illustrated in FIG. 3, a multi-array substrate may be contacted with target, e.g., hybridized with target, and read to provide a number of data files (or a single file having data for) all of the arrays on the substrate. The encoded information provided by the array information features may be decoded and used to identify which data was derived from which array. The decoded information may simply state “array 1”, “array 2”, etc., to indicate the array.

Programming

The invention also provides programming for analysis of array data to provide information about an array. In general, positions (i.e., addresses) of the one or more array information features have been defined for an array, the subject programming may analyze data from the array to provide any information provided by binding of target to those elements. If information is obtained, the programming may, for example, convert the information (e.g., a binary code) into a human readable code (e.g., a word or number), and associate the human readable code with the data such that when a user views the data, the information may also be viewed.

Programming according to the present invention, i.e., programming that allows array information to be extracted from array data, as described above, can be recorded on computer readable media, e.g. any medium that can. be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above described methodology.

Accordingly, the invention also provides a computer readable medium for decoding encoded array information. This medium typically comprises information for decoding, e.g., translating, encoded array information obtained from a array having one or more array information features. In many embodiments, the information for decoding is in the form of a computer-readable file, e.g., a text file such as a table or the like. In general, the information for decoding indicates (directly or indirectly via a second file), which features are array information features, which method should be used to decode the data obtained from those features, which type of information is encoded, and which features represent which part (i.e., “bit”) of the code.

In many embodiments of the invention, the decoding information for an array is provided by the design file for that array. As is well established in the microarray arts, arrays are typically associated with a file, such as a table, that contains information about which probes are on the array, i.e., which probe is present at each feature of the array. This file is commonly referred to as a “design file” and is generally well known in the art. In most cases a design file typically contains a lookup table containing a list of feature identifiers and a corresponding list of probe identifiers. The feature identifiers are typically numerical identifiers, e.g., 1, 2, 3, 4, etc., and correspond to the individual features of an array. The probe identifiers indicate the probe that is present in each feature. Typically, a probe identifier is a unique identifier that that can be used to query a database of probe information. Such design files are typically shipped with arrays that are purchased or may be obtained from a remote location. Typically, an array is associated with a particular design file using a unique identifier that is physically associated with the array (e.g., a bar code).

In many embodiments therefore, a design file for an array containing array information features may contain information to decode information obtained from those features. For example, in one embodiment, a design file will indicate which feature identifiers correspond to array information features, which code is being used, and which bit (part) of the code the feature represents. Without wishing to limit the invention, one aspect of the invention is shown in Table 1. Table 1 may represent part of a larger design file or the entire file. A in table 1 indicates that the features 1, 2, 3 and 4 are array information features, whereas B and C indicate the code used and the digit of the code respectively. C1 indicates that Feature ID No. 1 corresponds to the first digit of a code, and C2 indicates that Feature ID No. 2 corresponds to the second digit of the code, etc. A, B and C may be in any order. In certain embodiments, element D may also be present with elements A, B, and C to indicate the type of information that is being encoded.

TABLE 1
an exemplary design file

| Feature ID | Probe ID |
|---|---|
| 1 | A-B-C1 |
| 2 | A-B-C2 |
| 3 | A-B-C3 |
| 4 | A-B-C4 |

Depending on how A, B and C are indicated (e.g., if they are indicated using human readable words) they may be read manually or read by a computer and used to decode the information obtained using those features.

In alternative embodiments, a design file may indicate, at any position in the file, a second file, e.g., another table or executable program, that may be used to identify and decode the encoded information. In the example shown in Table 2, the tag “Decode using VI”, indicates that the encoded information may be decoded using “V 1”. V1 is a file that identifies particular features as array information features, and which method should be used to decode the data produced by those features, which type of information is encoded, and which features represent bits of the code. In certain embodiments V1 may be executable software for decoding information, for example.

Table 2: an exemplary design file, where W, X, Y and Z may be blank fields, may contain the tag “Decode using V1” or may contain any other type of information about the probe represented in those features.

Decode using V1

| Feature ID | Probe ID |
|---|---|
| 1 | W |
| 2 | X |
| 3 | Y |
| 4 | Z |

In certain embodiments, a design file may contain only probe information for array information features.

In use, a data file obtained from a scan of an array, e.g., a raw or processed data file, is typically linked to the above described information for decoding that data file. As is well known in the art, the data file typically includes evaluations of fluorescence intensity data for each element of an array. A data file may be linked to the correct decoding information by many methods, including by using a lookup table having lists of corresponding unique identifiers, e.g., filenames, barcodes, etc. Once linked, decoding software is typically executed, and the software reads the decoding information to identify which features are array information features, which method should be used to decode the data associated with those features, which type of information is encoded, and which features represent bits of the code. The software then assesses the data associated with the array information features and decodes the encoded information. In certain embodiments, the encoded information may be decoded without any other input information. However, in other embodiments, the encoded information is encoded using a database of codes. For example, if a binary code is used, the code may be looked up in a database to identify what is encoded by the code. In certain embodiments, therefore, decoding software may assess the data associated with a set of features to provide a code and compare the code to a database of codes to decode the code. In certain embodiments, the output of the decoding software may be used to annotate the data file decoded to provide an output file containing data and information about the array from which the data was obtained. In certain other embodiments, particularly those in which the design file used only contains information for array information features, the output of the decoding software may be used to indicate a further design file to be used in data analysis. In these embodiments, the further design file usually contains probe identifiers for non-array information features. In this embodiment, the array information features of an array effectively operate as a “molecular barcode”. Once read and decoded, the data obtained from those array features may be used to obtain a design file containing information for non-array information features on the array. This information could be obtained from a remote location.

FIG. 3 shows an exemplary embodiment of the invention: a data file 102 and decoding information 104 are linked 106. Data analysis software decodes the information encoded in the data file 108 to provide an output 110, which, in some embodiments is used to annotate the data file. The output may also be used to obtain a probe information corresponding to the data.

Such programming could be used in conjunction with or may be readily incorporated into any features extraction or any data analysis program. Several commercially available programs perform data analysis of microarrays, such as IMAGENE™ by BioDiscovery (Marina Del Rey, Calif.) Stanford University's “ScanAlyze” Software package, Microarray Suite of Scanalytics (Fairfax, Va.), “DeArray” (NIH); PATHWAYS™ by Research Genetics (Huntsville, Ala.); GEM tools™ by Incyte Pharmaceuticals, Inc., (Palo Alto, Calif.); Imaging Research (Amersham Pharmacia Biotech, Inc., Piscataway, N.J.); the RESOLVER™ system of Rosetta (Kirkland, Wash.) and the Feature Extraction Software of Agilent Technologies (Palo Alto, Calif.). Such commercially available programs may be adapted or modified to perform the subject methods.

Kits

Kits for use in connection with the subject invention are also provided. Such kits usually include one or more array information probes, and/or labeled target that binds to the one or more array information probes under specific binding conditions to provide information about an array. In certain kits, the one or more array information probes may be present in one or more array information features on an array, as discussed above. In particular embodiments, a subject kit may contain a set of array information targets for providing information on how data obtained from an array may be analyzed. For example, a kit may contain a set of array information targets that, when bound to a set of array information targets present on an array, conveys information to data analysis software on how data obtained from an array may be analyzed. Once array information is obtained, data analysis software, in view of the information, may analyze data obtained from an array in a particular way. For example, such targets may indicate which diseases or conditions an array may be used to investigate or diagnose. That information may be used by data analysis software to analyze data obtained from that array to obtain information about any or all of those diseases. Kits may also contain instructions for using the kit to produce at least one signal from at least one of the one or more array information probes to provide information about an array using the methods described above. In certain other embodiments, a subject kit may contain, sometimes in addition to the above kit components, a computer-readable medium containing information for decoding encoded information obtained from an array containing array information features. Accordingly, a subject kit may contain an array comprising array information features, and, instructions for obtaining information for decoding encoded array information encoded by those array information features. In certain embodiments, the instructions are for obtaining information from a remote location.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed from or from where the instructions can be downloaded. Still further, the kit may be one in which the instructions are obtained are downloaded from a remote source, as in the-Internet or world wide web. Some form of access security or identification protocol may be used to limit access to those entitled to use the subject invention. As with the instructions, the means for obtaining the instructions and/or programming is generally recorded on a suitable recording medium.

### EXPERIMENTAL

### Example 1

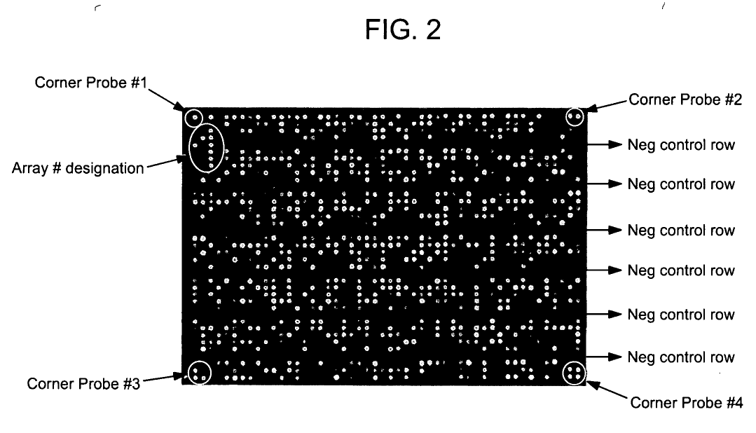

A system of targets, probes and labeling techniques may be used to encode non-biological information into a microarray, using, for example, binary labeling techniques. The binary code may be represented by the presence or a single label (i.e., a radioactive or non-radioactive label), or by the presence of one or two distinct distinguishable labels (e.g., generated Cy-3 or Cy-5). By extension, the system may be used to encode an alphabet of greater than 2 symbols where the normalized intensity of a color may represent unique, distinguishable symbols (i.e., 10 intensity levels could represent digits 0-9, twenty six intensity levels could represent the letters A-Z, etc.). Positive and negative control probes can also be laid out on the microarray to display a symbol that can be human readable, such as number, letter, graphic icon, etc. FIG. 2 shows an image of a single array of a multi-array substrate, hybridized with a labeled probe. The hybridization pattern provides non-biological information about the array. For example, in each corner of this array, signals from a set of four probes form a specific pattern that indicates the four corners of the array (i.e., a signal from the top left hand probe of the quartet of probes indicates the top left hand comer of the array; signals from the top left and top right hand probes of the quartet indicate the top right hand corner of the array; signals from all but the top right hand probes of the quartet indicate the bottom left corner of the array, and signals from all four probes indicate the bottom right corner of the array. Also shown in this figure is a subarray number, i.e., a designation that distinguishes one array of a multi-array substrate from other arrays of the same substrate. Typically these arrays are labeled 1-8. In the embodiment shown in FIG. 2, the array is designated with by the numeral “1”, written in dot matrix, beneath the top left hand corner of the array.

### Example 2

In this example, data from a multi-array substrate containing array information feathers is decoded to indicate the array from which the data was obtained.

Each array of a multi-array substrate containing eight arrays on a single slide is hybridized with a different sample. Data is obtained from this substrate by scanning the slide to make an image of the slide, and dividing the image into eight smaller images, each representing an individual array. Each of those smaller images is processed to provide eight files of data.

In order to indicate which file of data corresponds to which array, four features are used, in the case features 3, 4, 5, and 6. Each of the features either produce a signal, or do not produce a signal (depending on the probe composition present in each of the features or the sample hybridized to each of the arrays), to produce a binary coded decimal.

In this example, for each of the arrays, the following data is obtained, where “+” indicates a significant signal and “−” indicates a background signal:

| Array | Feature 3 | Feature 4 | Feature 5 | Feature 6 |
|---|---|---|---|---|
| number | signal | signal | signal | signal |
| 1 | + | − | − | − |
| 2 | − | + | − | − |
| 3 | + | + | − | − |
| 4 | − | − | + | − |
| 5 | + | − | + | − |
| 6 | − | + | + | − |
| 7 | + | + | + | − |
| 8 | − | − | − | + |

The design file for this array contains the following information:

| Feature | Probe |
|---|---|
| identifier | identifier |
| 3 | Encoded-ArrayIndex-BCD-Bit0 |
| 4 | Encoded-ArrayIndex-BCD-Bit1 |
| 5 | Encoded-ArrayIndex-BCD-Bit2 |
| 6 | Encoded-ArrayIndex-BCD-Bit3 |

The array data analysis software scans the design file for the word “Encoded” to identify array information features and to indicate that the software should decode information from the data for these features. The next keyword “ArrayIndex”, indicates to the software that the encoded information relates to the array number (in this case, the Arabic numerals 1-8 are indicated using a binary coded decimal code). The next word “BCD” indicates to the software that the type of encoded information is coded using the binary coded decimal system, and the “Bit” number indicates to the software how to group the information from the indicated features to form a single value, in this case, a binary coded decimal.

This binary coded decimal may be used to annotate the data file with the array from which the data is obtained. In certain embodiments, the binary coded decimal may be converted into an Arabic numeral before it is entered into the data file. In certain other embodiments, the binary coded decimal may be compared to a lookup table of database of binary coded decimals to identify the Arabic numeral it represents.

In another exemplary embodiment, the design file used for analysis may be indicated with the tag “EncodingVersion1”. This word provides a link to decoding information, and is recognizable by analysis software. Once recognized, a particular program (arbitrarily named “version 1” in this example) that contains information about which features are array information features, which method should be used to decode the data associated with those features, which type of information is encoded, and which features represent bits of the code, is executed to decode the encoded information.

In another exemplary embodiment, the design file used for analysis does not contain probe information for any features other than array information features 3, 4, 5, and 6. Once the array number has been determined by decoding the data for features 3, 4, 5 and 6, a design file containing probe information for all of the features is obtained automatically, and usually from a remote location, and linked to the data.

It is evident from the above discussion that the subject invention provides an important breakthrough in the labeling of arrays. Specifically, the subject invention allows one to encode information about the array on an array rather than on the label associated with a substrate containing the array. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A computer-readable medium comprising:
information for decoding encoded array information obtained from an array comprising one or more array information features.

2. The computer readable medium of claim 1, wherein said array is an array of nucleic acids.

3. The computer-readable medium of claim 1, wherein said information comprises a table that contains:
a list of feature identifiers; and
a list of probe identifiers corresponding to said feature identifiers.

4. The computer-readable medium of claim 3, wherein said table indicates that certain features of said array are array information features.

5. The computer readable medium of claim 3, wherein said table indicates which features correspond to which bit of a code.

6. The computer-readable medium of claim 1, wherein said information indicates an executable program for decoding said encoded array information.

7. The computer-readable medium of claim 1, wherein said information is a file that has a unique identifier that corresponds to a unique identifier of an array.

8. The computer-readable medium of claim 1, wherein said array information features encode binary coded information, and said file contains information for decoding said binary coded information.

9. The computer-readable medium of claim 7, wherein said binary coded information is encoded using a binary coded decimal (BCD) or binary ASCII code.

10. The computer-readable medium of claim 1, wherein said decoded array information indicates a particular array of a multi-array substrate.

11. A method for obtaining information about an array, comprising:
reading an array comprising one or more array information features to provide encoded information for said array; and
decoding said encoded information using a computer readable medium of claim 1 to provide information about said array.

12. The method of claim 11, wherein said information about said array indicates identifies said array as particular array of a multi-array substrate.

13. The method of claim 11, wherein said array is a nucleic acid array.

14. The method of claim 11, wherein said scanning provides a data file comprising feature identifiers and numerical assessments of the brightness of said array information features.

15. The method of claim 11, wherein said decoding comprises
identifying an executable program using said file, and
executing said program,
to decode said encoded information and provide information about said array.

16. The method of claim 15, wherein said executable program is obtained from a remote location.

17. A method for obtaining information about an array, comprising:
encoding information on an array using one or more array information features; and
providing information for decoding said encoded information.

18. The method of claim 17, wherein said array is shipped in the absence of said information.

19. The method of claim 17, wherein said array information is provided from a location remote to said array.

20. A method of assaying a sample, said method comprising:
(a) contacting said sample with an array comprising one or more array information features,
(b) reading said array with an array scanner to obtain data, and
(c) decoding said data using a computer readable medium of claim 1 to provide information for said array.

21. The method of claim 20, wherein said information for said array indicates that the data was derived from a particular array of a multi-array substrate.

22. The method according to claim 20, wherein said array is a nucleic acid array.

23. A method comprising transmitting a result obtained from a method of claim 20 from a first location to a second location.

24. The method of claim 23, where said second location is a remote location.

25. A method comprising receiving data representing said data obtained by the method of claim 20.

26. A kit for use with an array scanner, said kit comprising:
(a) a computer-readable medium according to claim 1; and
(b) instructions for operating said scanner according to said programming.

27. The kit of claim 26, further comprising an array.

28. A kit for use with an array scanner, said kit comprising:
(a) an array comprising array information features; and
(b) instructions for obtaining information for decoding encoded array information encoded by said array information features.
